(19) United States
(12) Patent Application Publication
(10) Pub. No.: US 2025/0127391 A1
(43) Pub. Date: Apr. 24, 2025

(54) 3D VISUAL ACUITY TEST METHOD USING BRAIN WAVES

(71) Applicant: Do Yeon PYO, Goyang-si (KR)

(72) Inventors: Do Yeon PYO, Goyang-si (KR); Ki Seong KIM, Daejeon (KR)

(21) Appl. No.: 18/492,955

(22) Filed: Oct. 24, 2023

(51) Int. Cl. A61B 3/028; A61B 5/00; A61B 5/11; A61B 5/378

(52) U.S. Cl. CPC A61B 3/028; A61B 5/1103; A61B 5/378; A61B 5/743; A61B 5/7475

(57) ABSTRACT

The present invention provides a 3D visual acuity test method, including: (a) making a subject cognize how to solve a problem; (b) providing the subject with a 3D image including the problem; and (c) receiving an answer to the problem from the subject, wherein steps (b) and (c) are performed together with measuring the brain waves of the subject, and a time from when the 3D image is provided in step (b) to when a cognitive peak for the answer to the problem appears in the brain waves is measured and used as data for determining the 3D visual acuity.

### BACKGROUND

### (a) Technical Field

The present invention relates to a 3D visual acuity test method using brain waves.

### (b) Background Art

In general, a person's visual function, visual acuity, indicates how small an object may be perceived or how small differences may be distinguished.

Recently, as the proportion of 3D content increases, interest in stereoscopic vision is also increasing. When a person is stereoscopic blindness that he/she cannot view a 3D shape at all or have low 3D visual acuity, it is not only difficult to view 3D content properly, but also difficult or impossible to achieve a goal in ball sports, driving a car, piloting an airplane, work that requires precise eye-hand coordination, mechanical engineering work, modeling work, etc. Therefore, a process of measuring accurate 3D visual acuity to confirm future occupational aptitude and work ability through stereoscopic vision perception may be used as very important data in choosing a suitable future.

The stereoscopic vision refers to a function of performing comprehensive determination on a 3D shape of an object using two eyes. There are various methods of a person to cognize a 3D shape. The most preferred method of cognizing a 3D shape is using binocular disparity. In addition, a sense of touch, perspective, use of light direction and shadow, comparison of sizes of objects, identification of familiar objects, hearing and echo, etc., may be used.

The 3D shape or 3D structure of the object may be cognized using only the sense of touch without visual information, and the 3D shape may also be identified by comparing a degree of size reduction according to the perspective of the object. The 3D shape of the object may also be estimated by identifying the direction of light shining on the object and shadow that appears on an opposite side of the object on which light shines. It is also possible to estimate the perspective of the object by comparing sizes of objects of the same type, and it is possible to estimate a 3D shape when the familiar object is already in the field of view. In addition, a method of cognizing a 3D shape by using sound and using an utterance point and echo of sound may be used.

The most typical method of a person to cognize a 3D shape is a method of using binocular disparity. A person's two eyes are designed to be able to view a video with a slight parallax to the left and right of an object, and thus, cognize a 3D shape through the process of interpreting the binocular disparity through the brain. When a person looks at an object, the closer a person looks at an object, the larger the binocular disparity becomes, and the more a person looks at a distant object, the smaller the binocular disparity becomes. FIG. 3 is a diagram briefly illustrating this and illustrates the binocular disparity in the case of viewing a distant object and in the case of viewing a close object when the distance between the two eyes is constant.

Typically, since the distant object has less binocular disparity than the closer object, it is easier to cognize the 3D shape of the close object than to cognize the 3D shape of the distant object. Therefore, it is more difficult to compare the 3D shapes of the distant objects than to compare the 3D shapes of the close objects, which may be used as a reference to measure the 3D visual acuity by testing how precise the distinction is possible.

Since a person possesses various mechanisms for cognizing a 3D shape in addition to the binocular disparity, even a person who is unable to cognize the 3D shape due to the binocular disparity are often able to cognize the 3D shape, so his/her daily lives are not particularly disrupted. Therefore, when no special tests have been performed, it is difficult for a person to know for himself/herself whether he/she cognizes the 3D shape through the binocular disparity.

The 3D visual acuity test may be a process of checking whether there are any problems in the 3D shape cognitive process using the binocular disparity. The 3D image for measuring the 3D visual acuity is composed of a left eye video that enters a subject's left eye and a right eye video that enters a subject's right eye. The left eye video and the right eye video are videos in which parallax is formed to the left and right at a certain angle, as if a 3D image is formed through the person's left and right eyes. This video may be captured directly using a camera designed to capture the 3D image, or may be artificially generated using a computer program or the like, or the 3D image may be realized through demonstration, etc. Depending on the environment in which the image is displayed to the subject, photos, drawings, videos, demonstrations, etc., may be used together.

A variety of cameras may be used to generate the 3D image, which is described in detail in Patent Nos. 10-20102300000, 10-15961460000, etc. Adobe Photoshop, Fusion 360, Blender, etc., are known as computer programs that generate 3D images, and 3D shapes may be created through composition using various 3D image production programs. Of course, when implementing the 3D image through the demonstration, various shapes, tools, items, etc., may be used to suit the test situation.

The conventional 3D visual acuity test method includes the following steps:
(1) Informing a subject of a 3D visual acuity test method;
(2) Placing the subject at a predetermined distance in front of the display device or having the subject wear a wearable display device;
(3) Providing the subject with a 3D image containing the problem and simultaneously starting time measurement;
(4) Receiving an answer to a problem from the subject;
(5) Comparing the answer given by the subject with a correct answer, and evaluating 3D visual acuity based on a time taken to receive the correct answer when the answer is correct.

However, in the conventional 3D visual acuity test method, individual abilities unrelated to stereoscopic vision intervene between the time the subject cognizes the answer to the 3D image problem and the time the subject completes the submission of the answer to the problem, so significant measurement noise occurs during this process.

Specifically, the process from the moment the subject perceives the 3D image to the moment the answer is submitted takes place in a very short period of time, and during this time, the following steps occur inside the subject.
1) Perceiving a 3D image with two eyes;
2) Interpreting a meaning in the perceived 3D image;
3) Cognizing the meaning within the 3D image;
4) Cognizing the answer to the problem; and
5) Expressing the answer

However, in step 5), in addition to the subject's 3D visual acuity, individual abilities required to provide an answer intervene are intervened. Individual abilities intervening in this area include brain activity, commonly called agility, motor ability necessary for the process of coming up with an answer, and the like. Therefore, if these factors are not distinguished from pure 3D cognition ability, it becomes difficult to accurately measure the 3D visual acuity.

Therefore, there is a need to develop a method that can solve these problems and measure accurate 3D visual acuity.

### RELATED ART DOCUMENT

### Patent Document

Korean Patent No. 10-1811890

### SUMMARY OF THE DISCLOSURE

The present invention has been made to solve the problems of the related art as described above.

The present invention provides a 3D visual acuity test method using brain waves capable of providing accurate 3D visual acuity measurement results by removing noise caused by individual abilities unrelated to 3D visual acuity that occurs when measuring the 3D visual acuity.

To achieve the object, the present invention provides a 3D visual acuity test method, including:
(a) making a subject cognize how to solve a problem;
(b) providing the subject with a 3D image including the problem; and
(c) receiving an answer to the problem from the subject,
wherein steps (b) and (c) are performed together with measuring the brain waves of the subject and the time, and
a time from when the 3D image is provided in step (b) to when a cognitive peak for the answer to the problem appears in the brain waves is measured and used as data for determining the 3D visual acuity.

According to a 3D visual acuity test method of the present invention, it is possible to provide highly accurate measurement results by completely removing measurement noise caused by the conventional test method, that is, noise caused by individual abilities unrelated to stereoscopic vision that intervenes between the time the subject cognizes an answer to a problem and the time the answer to the problem is submitted.

### BRIEF DESCRIPTION OF THE DRAWINGS

FIG. 1 is a flowchart illustrating a 3D visual acuity test method of the present invention.

FIG. 2 is a diagram illustrating an attachment position of an electrode for measuring brain waves during the 3D visual acuity test of the present invention.

FIG. 3 is a diagram illustrating binocular disparity according to a distance to an object when a distance between two eyes is constant.

FIG. 4 is a graph illustrating power spectrum used in analysis of brain waves.

FIG. 5 is a diagram illustrating an example of evoked potentials measured by event-evoked potentials.

FIG. 6 is a diagram illustrating another example of the evoked potentials measured by the event-evoked potentials.

FIG. 7 is an example of a 3D image created by arranging an image for left video and an image for right video with left and right binocular disparity left and right in one photo.

FIG. 8 is an image created by removing binocular disparity from the image with the binocular disparity in FIG. 7 to create a plane image without binocular disparity.

FIG. 9 is a diagram for describing a display detection position of a 3D image according to a position where subject's two eyes are aligned.

FIG. 10 is an example of a VR device that displays a 3D image on the left and right respectively.

### DETAILED DESCRIPTION

Hereinafter, preferred embodiments of the present invention will be described in detail with reference to the accompanying drawings. Before explaining the present invention, if a detailed description of related known functions and configurations is judged to unnecessarily obscure the gist of the present invention, the description thereof will be omitted. In addition, the terms and words as used herein are not to be construed as being limited to general or dictionary meanings, and are to be construed as meaning and concepts meeting the technical idea of the present invention based on a principle that the present inventors may appropriately define the concepts of terms in order to describe their own inventions in the best manner.

The description and drawings below illustrate specific embodiments to enable those skilled in the art to easily practice the devices and methods described. Other embodiments may include other structural and logical variations. Individual components and functions may be selected generically, unless explicitly required, and the order of processes may vary. Portions and features of some embodiments may be included in or replaced with other embodiments.

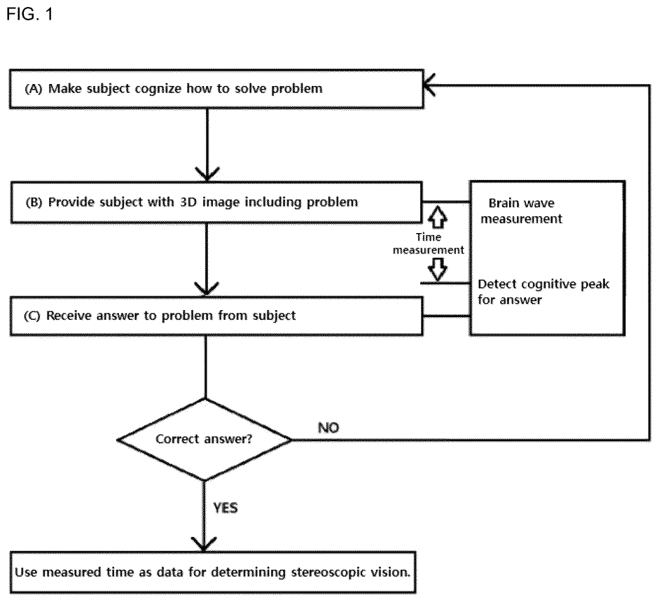

As illustrated in FIG. 1, a 3D visual acuity test method of the present invention includes:
(a) making a subject cognize how to solve a problem;
(b) providing the subject with a 3D image including the problem; and
(c) receiving an answer to the problem from the subject,
in which steps (b) and (c) are performed together with measuring the brain waves of the subject, and a time from when the 3D image is provided in step (b) to when a cognitive peak for the answer to the problem appears in the brain waves is measured and used as data for determining the 3D visual acuity.

The conventional 3D visual acuity test method measures the time from when a 3D image is provided to a subject to when an answer to a question is received from the subject, and evaluates the 3D visual acuity based on the measured amount of time.

However, between the time the 3D image is provided to the subject and the time the answer to the question is received from the subject,
(i) the time until a subject's brain cognizes the answer to the problem and
(ii) the time from the cognition of the above answer to the submission of the above answer are included.

Among the time (i) and (ii) above, the time (i) is a time directly related to the 3D visual acuity, but the time (ii) is a time required to submit the answer to the question and is directly unrelated to the 3D visual acuity. Furthermore, the time (ii) is unrelated to the 3D visual acuity and is a time determined by individual's abilities such as brain activity commonly called agility and motor ability required for a process of giving an answer, and thus, may be determined as noise in the 3D visual acuity measurement.

In particular, since the summed time of the (i) and (ii) is very short, when the time (ii) is included as noise, the accuracy of the 3D visual acuity measurement is greatly reduced. Therefore, in order to accurately measure the 3D visual acuity, the development of a noise removal method is essential.

The present inventors have made intensive efforts to increase the accuracy of the 3D visual acuity test, excluding the time (ii). The present invention was completed by discovering that, when the cognitive peak for the answer to the problem is detected by measuring brain waves during the 3D visual acuity test, it is possible to accurately measure the time, and when the time (i) is used as a standard for evaluating the 3D visual acuity, the accurate 3D visual acuity may be measured without noise.

### Brain Wave Measurement

In the present invention, brain wave measurement may be performed by generally known measurement methods. The brain wave measurement will be described in detail below.

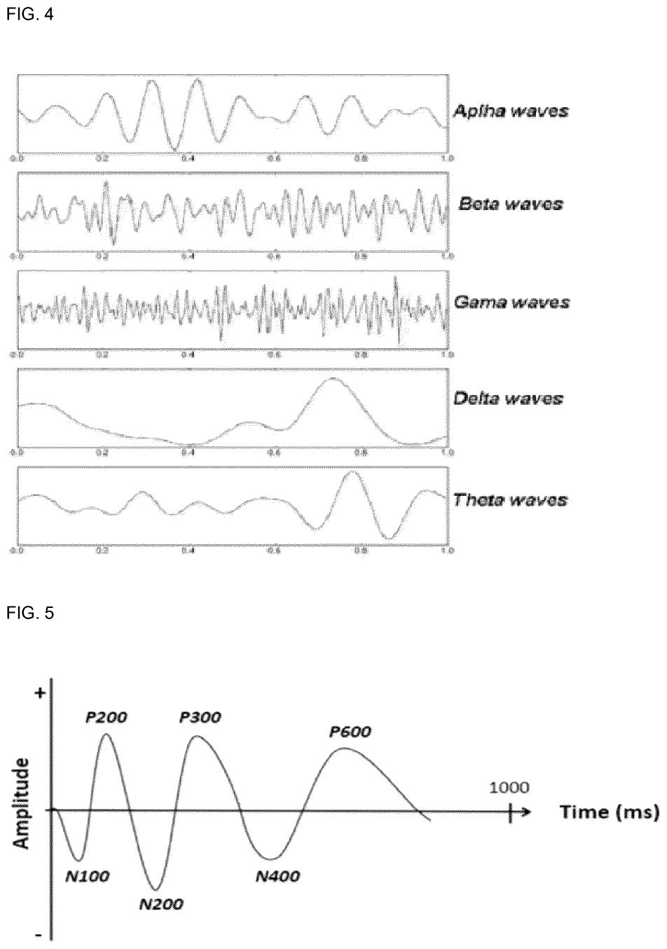

Typically, when measuring and analyzing person's brain waves, power spectrum analysis is used to classify the brain waves according to frequency. It is assumed that the power spectrum is a linear combination of simple vibrations that vibrate at a specific frequency, and in this signal, each frequency component is decomposed and its size is indicated. The types of brain waves using the power spectrum may be classified according to their frequency and amplitude when observing the brain waves. FIG. 4 is an example of the power spectrum graphically illustrated.

A wavelength of the brain waves coming from the human brain is generally 0 to 50 Hz and has amplitude of about 20 to 200 uV. The brain waves may be classified as follows.

Gamma wave: It has a frequency of 30 Hz or more and occurs relatively frequently in a frontal lobe and a parietal (central) lobe during extreme arousal and excitement.

Beta wave: It is a frequency of 13 to 30 Hz, and also called “stress wave.” It indicates activity waves such as anxiety and tension.

Alpha wave: It is a frequency of 8 to 12.99 Hz, and may be called a resting wave as a brain wave when the mind and body are at rest. It is a representative component of human brain waves and is known to be closely related to the development of the brain.

Theta wave: It is a frequency of 4 to 7.99 Hz and is called “drowsiness wave” or “slow-wave sleep wave.” It is known as a brain wave that passes through when falling asleep.

Delta wave: It is a frequency of 0.2 to 3.99 Hz, and also called “sleep wave.” It is known to occur during sleep.

Often, when trying to analyze brain wave characteristics of a specific state, the power spectrum distribution, which shows the overall power distribution for each frequency component of 0 to 50 Hz, is first measured and observed, and then the frequency components that change significantly may be found and referred to for analysis. The power spectrum distribution shows slightly different aspects for each measurement part of each channel. A cerebral cortex below a surface of a head may be largely divided into a frontal lobe, a parietal lobe, a temporal lobe, an occipital lobe, etc., and each region is known to have a different function. For example, it has been known that the occipital lobe is known to have a primary visual cortex, and thus, is responsible for processing primary visual information, and the parietal lobe near the crown of the head has a somatosensory cortex, and thus, is responsible for processing motor/sensory-related information.

In general, the method of presenting a certain stimulus or problem to a subject and then measuring a response to the stimulus through brain waves is called “event-evoked potential measurement.” Meaningful stimulation such as light stimulation, sound stimulation, or tactile stimulation is given to a subject, and changes in brain waves, which appear accordingly, are measured.

The brain waves may be largely divided into spontaneous potential (SP) and evoked potential (EP) components. The brain's neural network is constantly active regardless of external stimulation, and a waveform that measures the brain's electrical activity in real time is commonly called a brain wave. The component of the brain wave induced by the external stimulus is called the “evoked potential,” and is the opposite concept to the evoked potential, and the waveform that is distinct from this is called “spontaneous potential.”

The “evoked potential” refers to a waveform that presents a stimulus with specific information to a subject and then measures the electrical activity of the brain related to the stimulus processing. The “evoked potential” obtained after presenting the specific stimulus is measured together with the “spontaneous potential.” The evoked potential component obtained by presenting repetitive stimulation may be averaged and extracted based on the time when the stimulus was presented to remove the spontaneous potential component and extract only the evoked potential which is brain activity commonly involved in the stimulus processing. This averaging process is called ensemble averaging analysis.

Typically, the evoked potential may be represented as a picture of a simple waveform composed of a few peaks, and are preferred for diagnosis because it has better reproducibility and is analyzed relatively simpler compared to the spontaneous potential. To measure the evoked potential, the stimulus is presented periodically, and the time-specific peaks of the extracted waveform are analyzed and used for analysis related to various internal mental activities such as diagnosis of neurological problems, memory exploration, and a cognitive process.

Typically, peaks occurring at 50 ms to 200 ms (0.05 to 0.2 seconds) after stimulus presentation are mainly used in a neurology field to diagnose problems in a nerve transmission pathway, and peaks that occur between 200 ms and 800 ms (0.2 to 0.8 seconds) after the stimulus presentation reflect a lot of internal mental activities such as the attention, the memory exploration, and the cognitive process, and are mainly used in the fields of psychiatry and cognitive psychology. Depending on the researchers, the brain wave peak (peak between 50 ms and 200 ms) that primarily appears after the stimulus presentation as illustrated above is sometimes called a perceptual peak, and the brain wave peak (peak between 200 ms and 800 ms) that appears after the perceptual peak after the stimulus presentation is called the cognitive peak.

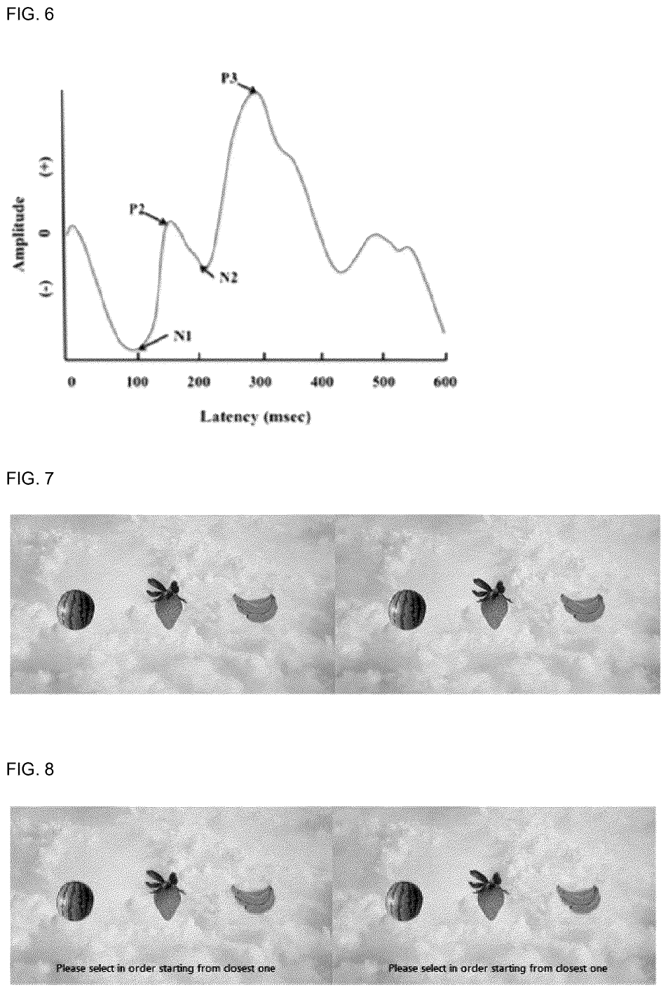

Among these peaks, much research has been conducted regarding the brain's information processing mechanism corresponding to P300. The P300 peak refers to a peak that appears around 300 ms after the stimulus presentation, and the peak usually appears between 250 ms and 700 ms depending on the properties of the stimulus and the individual differences. According to the previous researches, the P300 is known to reflect selective attention to stimulus, stimulus cognition, memory exploration, and resolution of uncertainty during the information processing process. In other words, it is known that the higher the attention, memory, cognitive ability, etc., the larger the amplitude of the P300 peak tends to be, and the time at which the P300 peak appears becomes faster. Usually, the P300 peak is also referred to as P3, and a peak of the P3 has the form illustrated in FIG. 6.

Among the brain waves related to the 3D visual acuity measurement, the cognitive peak may typically appear at 200 ms to 800 ms as described above, but the cognitive peak of the present invention may appear earlier or later depending on the cognitive difficulty of the 3D image displayed for the 3D visual acuity test, and the subject's physical and psychological state. Accordingly, it should be noted that the cognitive peak referred to in the present invention does not specifically refer to this time.

In an embodiment of the present invention, the brain wave measurement may be performed by measuring event-evoked potential.

In an embodiment of the present invention, the cognitive peak may be the evoked potential obtained by measuring the event-evoked potential.

In an embodiment of the present invention, the cognitive peak may be a peak that occurs between 200 ms and 800 ms (0.2 seconds and 0.8 seconds) after the 3D image is provided.

In an embodiment of the present invention, the cognitive peak may be the P300 peak.

In an embodiment of the present invention, the recognition peak may be a peak that has morphological similarity to a reference peak prepared by extracting the cognitive peak for the answer to the problem from the evoked potential obtained by measuring the event-evoked potential while repeatedly performing steps (b) and (c).

For example, when steps (b) and (c) are repeatedly performed on multiple subjects, a type of brain waves commonly found at the time of recognizing the answer to the problem may be discovered, and the cognitive peak may be extracted from the types of brain waves by removing noise. Therefore, this cognitive peak may be specified as a reference peak and used in the test method of the present invention.

In other words, when testing the 3D visual acuity, the test may be performed by specifying a peak similar to a previously prepared reference cognitive peak as the subject's cognitive peak.

In an embodiment of the present invention, the time from when the 3D image is provided in step (b) to when a perceptual peak for the answer to the problem appears in the brain waves is measured and used as auxiliary data for determining the 3D visual acuity.

In an embodiment of the present invention, the determination of the 3D visual acuity may be performed using measurement time data accumulated by a plurality of 3D visual acuity measurement results as a reference. For example, the determination of the 3D visual acuity may create a reference (for example, look up table) arranged by statistically processing the accumulated measurement time data, and express the 3D visual acuity in one or more selected from percentage and grade by comparing the time measured from the subject with the reference.

As a method of expressing 3D visual acuity with the above percentage (%), for example, based on the distribution of the 3D visual acuity within a preset population group, the 3D visual acuity of the subject may be displayed as the top or bottom percentage (%).

In addition, as a method of expressing 3D visual acuity with the above grade, for example, based on the distribution of the 3D visual acuity within a preset population group, the 3D visual acuity of the subject may be divided into 4 grades (high, middle, low, stereoscopic vision abnormality) and displayed as relative scores.

In addition, based on data measured from multiple subjects, the measured values may be averaged, and when the value is greater than or equal to the averaged value, it may be set as “+stereoscopic vision”, and when the value is less than or equal to the averaged value, it may be set as “-stereoscopic vision” to provide the result value.

In an embodiment of the present invention, when the answer received from the subject in step (c) is incorrect, a retest should be performed as illustrated in FIG. 1. In other words, when the answer submitted by the subject is incorrect, the peak that appears in the brain wave is also not a meaningful peak, so the measured time may not be used as it is. Therefore, in this case, it is necessary to repeat the test until the subject submits a reliable correct answer and use the time measured in the test for which a reliable correct answer is received as the basis for determining the 3D visual acuity.

In an embodiment of the present invention, step (b) may further include having the subject wear 3D glasses. In the test method of the present invention, when using a 3D image that may measure the 3D visual acuity without the 3D glass, the 3D glass is not necessary, but when using the 3D image in which the 3D image is recognized by the 3D glass, the 3D glass is required.

In an embodiment of the present invention, the 3D image in step (b) may be a printed 3D image, a 3D image or a 3D video displayed on a display device, and the test may be performed by demonstrating an object, etc., to the subject. Images or videos known in the field may be used without limitation as the printed 3D image, the 3D image or 3D video displayed on a display device, the demonstration material, and the like.

The 3D video refers to a more realistic video that allows an object to be perceived as being at a different depth than a screen on which the video is presented a two-dimensional video by providing binocular disparity information which is a difference in positions of videos projected to each eye. FIG. 7 is an example of a 3D image that may be used for such a test, and is an example of an image created to have binocular disparity using a computer program.

In an embodiment of the present invention, before providing the 3D image in step (b), a plane image with binocular disparity removed from the 3D image to be provided to the subject may be first provided, and the plane image may be converted into the 3D image while the subject is viewing the plane image and provided. In this case, it is preferable because more accurate 3D visual acuity measurement results may be obtained.

Specifically, in the 3D visual acuity test method of the present invention, the subject is presented in advance, with, for example, problems such as “What is the color of the closest ball?” or “Which fruit is closest to the subject?” Then, the 3D image including a shape of an object that may be distinguished from the front to the back representing the problem is presented. In this process, before the 3D image is presented to the subject, a plane image with only the binocular disparity removed from the 3D image may be presented first. In addition, while presenting a plane image from which the binocular disparity is removed, brain waves while the plane image is provided may be measured at all times and referred to for analysis.

The 2D and 3D images may be printed images, or images or videos displayed on a display device. The subject cognizes the shape of the object illustrated in the 3D image presented above, distinguishes between the close shape and the far shape, and submits an answer.

In an embodiment of the present invention, the display device may be a stationary display device or a wearable display device. As the stationary display device, various types of display devices known in the field may be used without limitation, and the wearable display device includes, but is not limited to, a VR headset, and devices known in the art may be used without limitation.

However, in order to more accurately achieve the purpose of testing the 3D visual acuity, which is an object of this invention, a display in which a subject should go through the process of aligning left and right eyeballs toward a target in order to view the displayed 3D image is preferable.

For example, the is because, when the left video and right video including the binocular disparity are each displayed without the process of aligning each of the left and right eyeballs toward the target, the accuracy of the 3D visual acuity measurement may be lowered in subjects with decreased 3D visual acuity due to strabismus, double vision, etc.

In general, when 3D monitors, 3D TVs, printed 3D images, demonstration materials, etc., are used for the 3D visual acuity test, during the process of the subject perceiving the 3D image from the moment the 3D image is displayed, through the process of aligning the left and right eyes toward the target (this target includes the above symbols, pictures, shapes, etc.), the target is focused and the 3D shape is perceived. Therefore, the display may provide results in which the degree of decrease in the 3D visual acuity due to strabismus, double vision, etc., is reflected in the measurement process.

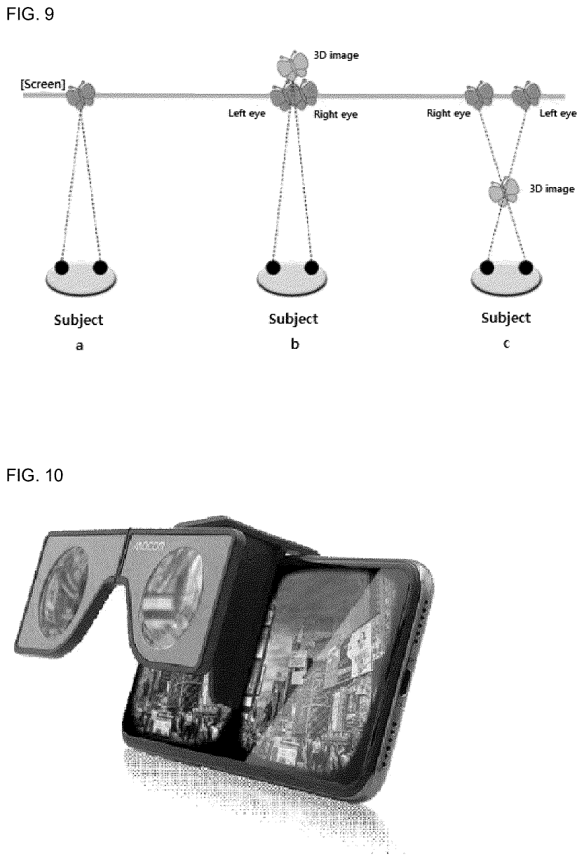

However, in the case of some displays, especially head-mounted displays such as VR devices, 3D images may be displayed simultaneously to the left and right eyes without going through the alignment process of the left and right eyes. Therefore, when using such displays, by creating a process so that the subject's eyes may focus on the target through an alignment process before displaying the 3D images or paying attention to the target arrangement of the 3D images and ensuring that the subject's eyes reach the target through the alignment process, the accurate test is possible. A detailed description of this part is given with reference to FIG. 9 as follows.

Subject a in FIG. 9 views a butterfly image displayed on the screen with both left and right eyes at the same time and in the same position. In this case, the left and right eyes of the subject a are aligned at the same position toward the target butterfly. When the left and right eyes are aligned in the same position as the screen, the subject perceives that the butterfly is on the same plane as the screen.

A left eye of subject b in FIG. 9 is aligned with the shape of the left butterfly displayed on the screen, and the right eye is aligned with the shape of the right butterfly displayed on the screen. In this case, the subject b feels that the butterfly is displayed further away from the screen.

A left eye of subject c in FIG. 9 is aligned with the shape of the right butterfly displayed on the screen, and the right eye is aligned with the shape of the left butterfly displayed on the screen. In this case, the subject c feels that the butterfly is displayed closer to the screen.

As shown in each subject case, in order to see the position of the target, the subject goes through the process of aligning the left and right eyes according to each situation and views a 3D shape. However, as illustrated in FIG. 10, in head-mounted displays such as VR devices where the left image of the 3D image and the right image of the 3D image are displayed separately to the two eyes, the subject's left and right eyes may view the 3D image without a separate alignment process. Therefore, care should be taken to artificially align two eyes or create and display the 3D image in which the two eyes may go through the alignment process.

In an embodiment of the present invention, in step (c), the answer to the problem may be submitted verbally by the subject, by pressing a button, or by touching the screen.

In an embodiment of the present invention, in order to increase accuracy of the measurement, the brain wave measurement may be performed by presenting a video that attracts a subject's attention or by providing compensation for a subject's concentration.

In an embodiment of the present invention, step (a) or step (b) may further include fixing electrodes for measuring brain waves to a scalp.

In an embodiment of the present invention, the method of measuring brain waves may be performed by a generally known method. For example, it may be used to measure brain waves while a plurality of electrodes (sensors) for measuring brain waves are fixed to the scalp.

The electrode unit for measuring brain waves may include a plurality of signal electrodes and reference electrodes. As the electrodes for measuring brain waves, plate electrodes or disposable adhesive electrodes may be used.

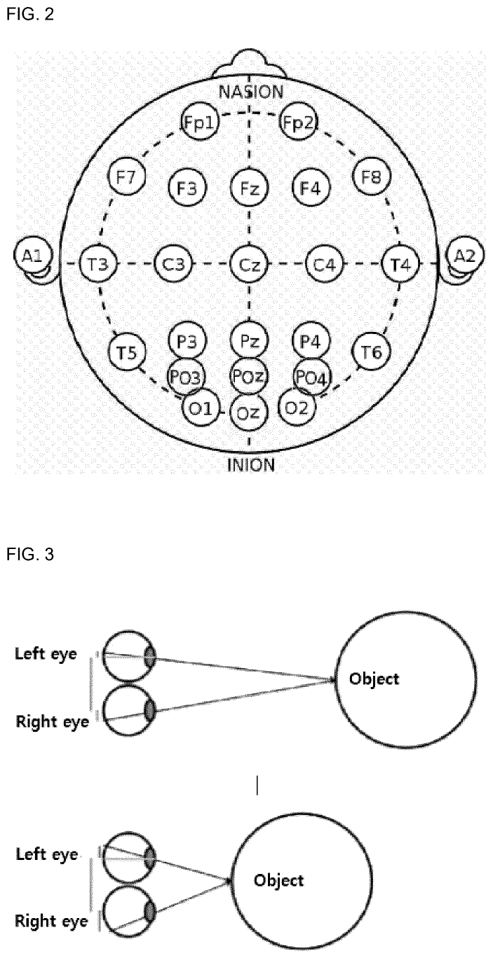

As illustrated in FIG. 2, electrodes may be attached to POZ, PO3, PO4, OZ, O1, and O2 on the subject's scalp according to the international 10-20 electrode arrangement method, and reference electrodes may be attached to A1 and A2 (both ear cheeks or below ears). These electrodes are attached to the occipital lobe and detect brain waves by measuring the flow of electricity generated when signals are transmitted between cranial nerves in the nervous system.

When the subject has low 3D visual acuity or is stereoscopic blindness, for the purpose of acquiring information that may help find the cause of the decrease in the 3D visual acuity, a sensor or camera that may acquire the subject's eye direction, pupil movement, facial direction information, etc., may be used together.

While the subject goes through the 3D visual acuity test, a sensor or camera is installed for the purpose of recording the subject's facial direction and the position of the pupils, such as on the subject's face and frontal position, on the 3D glasses, or on the display monitor, may be used as a means of obtaining auxiliary information for the 3D visual acuity measurement. In this case, the position of the camera or sensor is not specific, and it may be used to acquire the information by determining either the position of the 3D image display or the position that is the reference point for the position of the subject's pupil.

For example, when the subject is suffering from the decrease in the 3D visual acuity due to the strabismus, there may be a problem in that the alignment direction of the subject's left or right pupil is not aligned straight toward the display. This may be recorded together during the 3D visual acuity test and used as information to help find the cause of decrease in the 3D visual acuity decline.

When simultaneously testing the positions of the subject's eyes using the camera, the direction and movement of the pupils may be recorded even before the 3D image is displayed, so that information such as the direction, blinking, and movement of the pupils may be acquired from the moment the 3D image is displayed.

In addition, by using the subject's eye blinking to determine the 3D image display point, the noise due to the subject's eye blinking may be prevented from occurring in the detected brain waves. For example, when the subject blinks his/her eyes irregularly from time to time, irregular noise may be mixed into the brain waves due to the eye blinking, so the eye detection sensor may also be used by using the subject's eye blinking as a trigger to delay a certain period of time and then displaying the 3D image.

What is claimed is:

1. A 3D visual acuity test method, comprising:
(a) making a subject cognize how to solve a problem;
(b) providing the subject with a 3D image including the problem; and
(c) receiving an answer to the problem from the subject,
wherein steps (b) and (c) are performed together with measuring the brain waves of the subject, and
a time from when the 3D image is provided in step (b) to when a cognitive peak for the answer to the problem appears in the brain waves is measured and used as data for determining the 3D visual acuity.

2. The 3D visual acuity test method of claim 1, wherein the brain wave measurement is performed by measuring event-evoked potential.

3. The 3D visual acuity test method of claim 1, wherein the cognitive peak is an evoked potential obtained by measuring the event-evoked potential.

4. The 3D visual acuity test method of claim 3, wherein the cognitive peak is a peak that occurs between 200 ms and 800 ms after the 3D image is provided.

5. The 3D visual acuity test method of claim 4, wherein the cognitive peak is a P300 peak.

6. The 3D visual acuity test method of claim 1, wherein the cognitive peak is a peak that has morphological similarity to a reference peak prepared by extracting the cognitive peak for the answer to the problem from evoked potentials obtained by measuring the event-evoked potential while repeatedly performing steps (b) and (c).

7. The 3D visual acuity test method of claim 1, wherein a time from when the 3D image is provided in step (b) to when a perceptual peak for the answer to the problem appears in the brain waves is measured and used as auxiliary data for determining the 3D visual acuity.

8. The 3D visual acuity test method of claim 1, wherein the determination of the 3D visual acuity is performed using measurement time data accumulated by multiple 3D visual acuity measurement results as a reference.

9. The 3D visual acuity test method of claim 8, wherein the determination of the 3D visual acuity creates a reference arranged by statistically processing the accumulated measurement time data, and expresses the 3D visual acuity in one or more selected from percentage and grade by comparing the time measured from the subject with the reference.

10. The 3D visual acuity test method of claim 1, wherein when the answer received from the subject in step (c) above is incorrect, a retest is performed.

11. The 3D visual acuity test method of claim 1, wherein step (b) above further comprises having the subject wear 3D glasses.

12. The 3D visual acuity test method of claim 1, wherein in step (b), the 3D image is one of a printed 3D image, a 3D image displayed on a display device, a 3D video, and a demonstration material.

13. The 3D visual acuity test method of claim 1, wherein before providing the 3D image in step (b), a plane image with binocular disparity removed from the 3D image to be provided to the subject is first provided, and the plane image is converted into the 3D image while the subject is viewing the plane image and provided.

14. The 3D visual acuity test method of claim 1, wherein in step (c), the answer to the problem is received verbally from the subject, or submitted by pressing a button, or by touching a screen.

15. The 3D visual acuity test method of claim 1, wherein in order to increase accuracy of the measurement, the brain wave measurement is performed by presenting a video that attracts a subject's attention or by providing compensation for a subject's concentration.

16. The 3D visual acuity test method of claim 1, wherein the 3D visual acuity test method additionally utilizes one or more sensors or cameras that measure and record a direction of a subject's pupil and face.

17. The 3D visual acuity test method of claim 16, wherein the sensor or camera includes a function of detecting subject's eye blinking.

18. The 3D visual acuity test method of claim 17, wherein the sensor or camera detects the subject's eye blinking to determine a time the 3D image is displayed.

19. The 3D visual acuity test method of claim 13, wherein the plane image from which the binocular disparity has been removed is either a left image or a right image of the 3D image to be presented to the subject.

20. The 3D visual acuity test method of claim 1, wherein in step (b), the display that provides the 3D image including the problem to the subject is a display in which the subject goes through an alignment process of two eyes to view the 3D image.
